(19) United States
(12) Patent Application Publication
(10) Pub. No.: US 2008/0113002 A1
(43) Pub. Date: May 15, 2008

(54) Contact lens compositions

(76) Inventors: Saul Yedgar, Jerusalem (IL); Yuval Cohen, New York, NY (US)

Correspondence Address: Pearl Cohen Zedek Latzer, LLP, 1500 Broadway, 12th Floor, New York, NY, 10036, US

(21) Appl. No.: 11/984,224

(22) Filed: Nov. 14, 2007

Related U.S. Application Data
(60) Provisional application No. 60/858,706, filed on Nov. 14, 2006.
(60) Provisional application No. 60/907,785, filed on Apr. 17, 2007.

(51) Int. Cl. A61K 9/00; A61P 27/00

(52) U.S. Cl. 424/429

(57) ABSTRACT

In one embodiment, the invention provides a contact lens comprising a lipid or phospholipid moiety bound optionally via a spacer to a physiologically acceptable monomer, dimer, oligomer, or polymer via an ester or amide bond, and/or a pharmaceutically acceptable salt or a pharmaceutical product thereof.

### FIELD OF THE INVENTION

This invention provides compounds and methods of use thereof for contact lenses comprising said compounds.

### BACKGROUND OF THE INVENTION

Man has attempted to correct and adapt his conditions to enjoy perfect vision, or to endow it with greater resources for centuries. Diverse instruments have been invented toward this end, including spectacles, microscopes, lenses, etc. with a primitive concept of a contact lens conceived by Leonardo da Vinci (1452-1519) and Rene Descartes (1596-1650), while the first definitive description of a contact lens, a glass capsule full of gel functioning as a posterior refractive surface, was published by the English astronomer John Frederick William Herschell, in the early 1800s.

Contact lenses as therapeutics were also described, including use of a lens soaked in mercury saline, to aid in the cure and reduction of infections arising from cataract surgery.

Other milestones that addressed the difficulties in the manufacturing of contact lenses included the production of contact lenses through a process of spin casting and the development of gas permeable lenses.

Currently there exist two principal types of lenses, soft and hard. Soft contact lenses absorb water, are flexible and plastic, and adhere to the surface of the cornea, and comprise conventional lenses as well as disposable lenses, lenses suitable for daily or extended wear; colored lenses, utilizable for daily or extended use, aesthetic lenses which serve to cover defects of the iris or pupil arising from infectious diseases or traumas to the eyes that cause eyes to be whitened or blemished; and removable bifocal and toric lenses.

Hard contact lenses are hard lenses that are not absorbent to water, yet are gas permeable. Like soft lenses, hard lenses can be used for daily or extended wear.

The various types of contact lenses possess points where dirt, bacteria, chemical products and eye secretion can accumulate themselves, and provoke, inter alia, an inflammatory reaction.

Over 29 million people in the United States wear contact lenses, which provide a safe and effective way to correct vision when used with care and proper supervision. However, many contact lens wearers and potential contact lens wearers suffer from discomfort, dry eyes, and infection as a result of contact lens use. There is therefore a need to design contact lenses to comprise compounds that allow the lens to be more biocompatible, comfortable, tear-wettable, anti-bacterial and oxygen permeable.

### SUMMARY OF THE INVENTION

In one embodiment, the invention provides a contact lens comprising a lipid or phospholipid moiety bound optionally via a spacer to a physiologically acceptable monomer, dimer, oligomer, or polymer via an ester or amide bond, and/or a pharmaceutically acceptable salt or a pharmaceutical product thereof.

In one embodiment, the lipid or phospholipid moiety bound optionally via a spacer to a physiologically acceptable monomer, dimer, oligomer, or polymer via an ester or amide bond, and/or a pharmaceutically acceptable salt or a pharmaceutical product thereof is represented by the structure of the general formula (A):

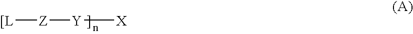

wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a physiologically acceptable monomer, dimer, oligomer, or polymer; and
n is a number from 2 to 1000;
wherein any bond between L, Z, Y and X is either an amide or an esteric bond.

In one embodiment, L is phosphatidyl, Z is ethanolamine, Y is nothing, and X is carboxymethylcellulose or a glycosaminoglycan.

In one embodiment, the phosphatidylethanolamine moiety is dipalmitoyl or dimyristoyl phosphatidylethanolamine.

In one embodiment, the lipid or phospholipid moiety bound optionally via a spacer to a physiologically acceptable monomer, dimer, oligomer, or polymer via an ester or amide bond, and/or a pharmaceutically acceptable salt or a pharmaceutical product thereof is represented by the structure of the general formula (I):

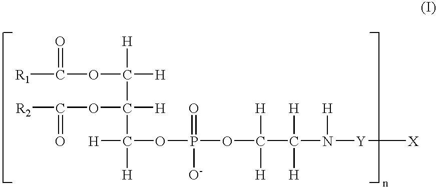

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms; and
Y is either nothing or a spacer group ranging in length from 2 to 30 atoms;
X is a physiologically acceptable monomer, dimer, oligomer, or polymer, and wherein X is a glycosaminoglycan; and
n is a number from 1 to 1000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

In one embodiment, n is a number from 2 to 100.

In one embodiment, wherein X is hyaluronic acid, heparin or chondroitin sulfate.

### DETAILED DESCRIPTION OF THE INVENTION

In one embodiment, this invention provides contact lenses and methods of utilizing the same.

In one embodiment, the lenses of this invention and methods of use thereof may find application in treating or preventing or treating secondary effects of any disease or disorder of the eye, which is positively affected by use of such lenses. In one embodiment, the lenses of this invention and methods of use thereof may find application in treating or preventing or treating secondary effects of: intraocular lens replacement; ophthalmic enucleation, evisceration, exenteration, or a combination thereof; lacrimal sac surgeries; corneal pterygium; lamellar keratoplasty; penetrating keratoplasty, or a combination thereof, as well as any of the disorders or conditions mentioned herein.

In one embodiment, the disease or disorder of the eye affects the anterior region of the eye, while in another embodiment, it affects the posterior region of the eye, while in another embodiment, it affects both the anterior and posterior regions of the eye. In one embodiment, the anterior segment includes the cornea, anterior chamber, iris and ciliary body (anterior choroid), posterior chamber and crystalline lens and the posterior segment includes the retina with optic nerve, choroid (posterior choroid) and vitreous. In one embodiment, eye disorders resulting from the pathologic conditions of structures in the anterior segment of the eye are dry eye syndrome, keratitis or corneal dystrophy, cataracts, and glaucoma. In one embodiment, the disease or disorders of the posterior segment of the eye in general are retinal or choroidal vascular diseases or hereditary diseases such as Lebers congenital amaurosis.

In one embodiment, “treating” refers to both therapeutic treatment and prophylactic or preventive measures, wherein the object is to prevent or lessen the targeted pathologic condition or disorder as described hereinabove. Thus, in one embodiment, treating may include suppressing, inhibiting, preventing, treating, or a combination thereof. Thus, in one embodiment, “treating” refers, inter alia, to increasing time to sustained progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers, inter alia, to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of an eye disease, while in one embodiment, “secondary” refers to a symptom that is derived from or consequent to a primary cause. In one embodiment, the compounds for use in the present invention treat primary or secondary symptoms or secondary complications related to an eye disease or disorder.

In another embodiment, “symptoms” may be any manifestation of a disease or pathological condition, comprising inflammation, swelling, fever, pain, bleeding, itching, runny nose, coughing, headache, migraine, dizziness, blurry vision, decreased visual acuity, light sensitivity, etc., or a combination thereof. In one embodiment, symptoms comprise itchy eyes, swollen eyelids, redness, irritation, watery eyes, mucoid discharge, pain, or a combination thereof.

Thus, in one embodiment of the present invention, the lenses for use in the present invention, comprising a compound as herein described, find application in the resolution of symptoms of a disease or disorder of the eye. In another embodiment, the lenses of this invention affect the pathogenesis underlying a disease or disorder of the eye.

In another embodiment, the methods of the present invention may be used to prevent or treat glaucoma. In one embodiment, glaucoma is characterized by increased fluid pressure in the eye, which in one embodiment, is due to slowed fluid drainage from the eye. In one embodiment, glaucoma may damage the optic nerve and other parts of the eye, lead to vision loss or blindness, or a combination thereof. In one embodiment, glaucoma may refer to primary open angle glaucoma, normal pressure glaucoma, normal tension glaucoma, pigmentary glaucoma, pseudoexfoliation glaucoma, acute angle closure glaucoma, absolute glaucoma chronic glaucoma, congenital glaucoma, juvenile glaucoma, narrow angle glaucoma, chronic open angle glaucoma, simplex glaucoma, primary congenital glaucoma, secondary glaucoma, or a combination thereof.

Administration of the compounds for use in the present invention in a diversity of animal and cell models of disease invoke remarkable, and unexpected, cytoprotective effects, which are useful in the prevention and treatment of eye diseases and/or conditions, which when incorporated in a contact lens, as herein described, impart such effects to the lens wearer.

In one embodiment of the present invention, the useful pharmacological properties of the compounds incorporated within the lenses of this invention, may be applied for clinical use, and disclosed herein as methods for the prevention or treatment of a disease. The biological basis of these methods may be readily demonstrated by standard cellular and animal models of disease, for example, as described in the Examples hereinbelow.

In one embodiment, the pharmacological activities of compounds for use in the present invention, including membrane stabilization, anti-inflammation, anti-oxidant action, and attenuation of chemokine levels, may contribute to the resistance to diseases of the eye in a wearer of a lens incorporating the compounds. In one embodiment, cell membrane stabilization may ameliorate or prevent tissue injury arising in the course of an eye disease. In another embodiment, anti-oxidant action may limit oxidative damage to cell and blood components arising in the course of an eye disease. In another embodiment, attenuation of chemokine levels may attenuate physiological reactions to stress that arise in the course of an eye disease.

This invention encompasses, in some embodiments, contact lenses comprising lipid conjugates.

In some embodiments, any of the contact lenses of this invention will comprise a lipid conjugate, in any form or embodiment as described herein. In some embodiments, the term “comprise” refers to the inclusion of the indicated active agent, such as the Compounds I-C, as well as inclusion of other active agents, and polymers, carriers, excipients, emollients, stabilizers, etc., as are known in the lens making industry.

In one embodiment, the lenses of the instant invention comprise a compound of the instant invention and additional compounds effective in preventing or treating eye disease. In one embodiment, the additional compounds comprise anti-inflammatory compositions, which in one embodiment are non-steroidal anti-inflammatory medications, antihistamines, antibiotics, corticosteroids, cromolyn sodium (sodium cromoglicate), mast-cell stabilizers, artificial tears, lubricants, or a combination thereof. In one embodiment, antibiotics comprise chloramphenicol, fusidic acid, tetracycline, erythromycin, gentamycin, or a combination thereof. In another embodiment, an additional compound is vitamin A.

In one embodiment, the lesnes of the instant invention comprise or are applied with other treatments that relieve symptoms.

In one embodiment, the lenses of this invention are used to treat an eye condition or disease.

In some embodiments, the lenses of this invention comprise lenses implanted within the eye.

In some embodiments, the lenses may have various dosages of the compounds to suit a particular application, as will be appreciated by the skilled artisan.

In general, the doses utilized for the above described purposes will vary, but will be in an effective amount to exert the desired effect. As used herein, the term “pharmaceutically effective amount” refers to an amount of a compound of formulae A and I-LXXXVII as described hereinbelow, which will produce the desired alleviation in symptoms or treatment of the disease or disorder or produce any other desired phenotype in a subject.

In one embodiment of the invention, the concentrations of the compounds will depend on various factors, including the nature of the condition to be treated, the condition of the patient, the route of administration and the individual tolerability of the compositions.

It will be appreciated that the actual preferred amounts of active compound in a specific case will vary according to the specific compound being utilized, the particular compositions formulated, the mode of application, and the particular conditions and organism being treated. Dosages for a given host can be determined using conventional considerations, e.g. by customary comparison of the differential activities of the subject compounds and of a known agent, e.g. by means of an appropriate, conventional pharmacological protocol.

In one embodiment, the present invention offers methods for the treatment of disease based upon administration of lipids covalently conjugated through their polar head group to a physiologically acceptable chemical moiety, which may be of high or low molecular weight.

### Compounds

In one embodiment, the compounds for use in the present invention or for incorporation in the lenses of the present invention comprise a lipid or phospholipid moiety bound to a physiologically acceptable monomer, dimer, oligomer, or polymer. In one embodiment, the physiologically acceptable monomer, dimer, oligomer, or polymer is salicylate, salicylic acid, aspirin, a monosaccharide, lactobionic acid, maltose, an amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate, a dipeptide, a disaccharide, a trisaccharide, an oligosaccharide, a polysaccharide, a hetero-polysaccharide, a homo-polysaccharide, a polypyranose, an oligopeptide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid, a glycosaminoglycan, polygeline (‘haemaccel’), alginate, hydroxyethyl starch (hetastarch), polyethylene glycol, polycarboxylated polyethylene glycol, chondroitin-6-sulfate, chondroitin-4-sulfate, keratin, keratin sulfate, heparan sulfate, dermatin, dermatan sulfate, carboxymethylcellulose, heparin, dextran, or hyaluronic acid.

In one embodiment, examples of polymers which can be employed as the conjugated moiety for producing the compounds for use in the present invention or for incorporation in the lenses of the present invention may be physiologically acceptable polymers, including water-dispersible or -soluble polymers of various molecular weights and diverse chemical types, mainly natural and synthetic polymers, such as glycosaminoglycans, hyaluronic acids, heparin, heparin sulfates, chondroitin sulfates, chondroitin-6-sulfates, chondroitin-4-sulfates, keratins, keratin sulfates, dermatins, dermatan sulfates, dextrans, plasma expanders, including polygeline (“Haemaccel”, degraded gelatin polypeptide cross-linked via urea bridges, produced by “Behring”), “hydroxyethylstarch” (Hetastarch, HES) and extrans, food and drug additives, soluble cellulose derivatives (e.g. methylcellulose, carboxymethylcellulose), polyaminoacids, hydrocarbon polymers (e.g. polyethylene), polystyrenes, polyesters, polyamides, polyethylene oxides (e.g. polyethyleneglycols, polycarboxyethyleneglycols, polycarboxylated polyethyleneglycols), polyvinnylpyrrolidones, polysaccharides, polypyranoses, alginates, assimilable gums (e.g. xanthan gum), peptides, injectable blood proteins (e.g. serum albumin), cyclodextrin, and derivatives thereof.

In one embodiment, examples of monomers, dimers, and oligomers which can be employed as the conjugated moiety for the compounds for use in the present invention or for incorporation in the lenses of the present invention may be mono- or disaccharides, trisaccharides, oligopeptides, carboxylic acids, dicarboxylic acids, fatty acids, dicarboxylic fatty acids, salicylates, slicyclic acids, acetyl salicylic acids, aspirins, lactobionic acids, maltoses, amino acids, glycines, glutaric acids, succinic acids, dodecanoic acids, didodecanoic acids, bile acids, cholic acids, cholesterylhemisuccinates, and di- and trisaccharide unit monomers of polysaccharides, polypyranoses, and/or glycosaminoglycans including heparins, heparan sulfates, hyaluronic acids, chondroitins, chondroitin sulfates, chondroitin-6-sulfates, chondroitin-4-sulfates, dermatins, dermatan sulfates, keratins, keratan sulfates, or dextrans.

In one embodiment, the lipid compounds for the compounds for use in the present invention or for incorporation in the lenses of the present invention are described by the general formula:

[phosphatidylethanolamine-Y]n-X

[phosphatidylserine-Y]n-X

[phosphatidylcholine-Y]n-X

[phosphatidylinositol-Y]n-X

[phosphatidylglycerol-Y]n-X

[phosphatidic acid-Y]n-X

[lyso-phospholipid-Y]n-X

[diacyl-glycerol-Y]n-X

[monoacyl-glycerol-Y]n-X

[sphingomyelin-Y]n-X

[sphingosine-Y]n-X

[ceramide-Y]n-X

wherein

### Y is either nothing or a spacer group ranging in length from 2 to 30 atoms; and

### X is a physiologically acceptable monomer, dimer, oligomer or polymer; and

n is the number of lipid molecules bound to a molecule of X, wherein n is a number from 1 to 1000. In another embodiment, n is a number from 2 to 1000.

In one embodiment, the invention provides low-molecular weight compounds, previously undisclosed and unknown to possess pharmacological activity, of the general formula described hereinabove. In another embodiment, wherein the general formula described hereinabove describes low-molecular weight compounds, X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid.

In one embodiment of this invention, X is any of the physiologically acceptable monomer, dimer, oligomer, or polymer, as described herein. In one embodiment, X is conjugated to the lipid, phospholipid, or spacer via an ester bond. In another embodiment, X is conjugated to the lipid, phospholipid, or spacer via an amide bond.

As defined by the structural formulae provided herein for the compounds for use in the present invention, these compounds may contain between one to one thousand lipid moieties bound to a single physiologically acceptable polymer molecule. In one embodiment of this invention, n is a number from 1 to 1000. In another embodiment, n is a number from 1 to 500. In another embodiment, n is a number from 1 to 100. In another embodiment, n is a number from 1 to 50. In another embodiment, n is a number from 1 to 25. In another embodiment, n is a number from 1 to 10. In another embodiment, n is a number from 1-5. In another embodiment, n is a number from 1 to 4. In another embodiment, n is a number from 1 to 3, In another embodiment, n is a number from 1 to 2. In another embodiment, n is a number from 2 to 1000. In another embodiment, n is a number from 2 to 200. In another embodiment, n is a number from 2 to 100. In another embodiment, n is a number from 2 to 50. In another embodiment, n is a number from 2 to 25. In another embodiment, n is a number from 2-10. In another embodiment, n is a number from 2 to 5. In another embodiment, n is a number from 2 to 4, In another embodiment, n is a number from 2 to 3. In another embodiment, n is a number from 3 to 300. In another embodiment, n is a number from 10 to 400. In another embodiment, n is a number from 50 to 500. In another embodiment, n is a number from 100 to 300. In another embodiment, n is a number from 300 to 500. In another embodiment, n is a number from 500 to 800. In another embodiment, n is a number from 500 to 1000.

In one embodiment of the invention, when the conjugated moiety is a polymer, the ratio of lipid moieties covalently bound may range from one to one thousand lipid residues per polymer molecule, depending upon the nature of the polymer and the reaction conditions employed. For example, the relative quantities of the starting materials, or the extent of the reaction time, may be modified in order to obtain products with either high or low ratios of lipid residues per polymer, as desired.

In one embodiment, the set of compounds comprising phosphatidylethanolamine covalently bound to a physiologically acceptable monomer, dimmer, oligomer, or polymer, is referred to herein as the PE-conjugates. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment, related derivatives, in which either phosphatidylserine, phosphatidylcholine, phosphatidylinositol, phosphatidic acid or phosphatidylglycerol are employed in lieu of phosphatidylethanolamine as the lipid moiety provide equivalent therapeutic results, based upon the biological experiments described below for the compounds for use in the present invention and the structural similarities shared by these compounds.

In another embodiment, the lipid or phospholipid moiety is phosphatidic acid, an acyl glycerol, monoacylglycerol, diacylglycerol, triacylglycerol, sphingosine, sphingomyelin, chondroitin-4-sulfate, chondroitin-6-sulfate, ceramide, phosphatidylethanolamine, phosphatidylserine, phosphatidylcholine, phosphatidylinositol, or phosphatidylglycerol, or an ether or alkyl phospholipid derivative thereof.

In one embodiment, derivatives relevant to this invention are compounds wherein at least one of the fatty acid groups of the lipid moieties at position C1 or C2 of the glycerol backbone are substituted by a long chain alkyl group attached by amide, ether or alkyl bonds, rather than ester linkages.

In the methods, according to embodiments of the invention, the compounds for use in the present invention administered to the subject are comprised from at least one lipid moiety covalently bound through an atom of the polar head group to a monomeric or polymeric moiety (referred to herein as the conjugated moiety) of either low or high molecular weight. When desired, an optional bridging moiety can be used to link the compounds for use in the present invention moiety to the monomer or polymeric moiety. The conjugated moiety may be a low molecular weight carboxylic acid, dicarboxylic acid, fatty acid, dicarboxylic fatty acid, acetyl salicylic acid, cholic acid, cholesterylhemisuccinate, or mono- or di-saccharide, an amino acid or dipeptide, an oligopeptide, a glycoprotein mixture, a di- or trisaccharide monomer unit of a glycosaminoglycan such as a repeating unit of heparin, heparan sulfate, hyaluronic acid, chondroitin-sulfate, dermatan, keratan sulfate, or a higher molecular weight peptide or oligopeptide, a polysaccharide, a hetero-polysaccharide, a homo-polysaccharide, a polypyranose, polyglycan, protein, glycosaminoglycan, or a glycoprotein mixture. The composition of some phospholipid-conjugates of high molecular weight, and associated analogues, are the subject of U.S. Pat. No. 5,064,817, which is incorporated herein in its entirety by reference.

In one embodiment, the term “moiety” means a chemical entity otherwise corresponding to a chemical compound, which has a valence satisfied by a covalent bond.

In some cases, according to embodiments of the invention, the monomer or polymer chosen for preparation of the compound may in itself have select biological properties. For example, both heparin and hyaluronic acid are materials with known physiological functions. In the present invention, however, the compounds for use in the present invention formed from these substances as starting materials display a new and wider set of pharmaceutical activities than would be predicted from administration of either heparin or hyaluronic acid which have not been bound by covalent linkage to a phospholipid. In some embodiments, phosphatidylethanolamine (PE) linked to hyaluronic acid (Compound XXII), to heparin (Compound XXIV), to chondroitin sulfate A (Compound XXV), to carboxymethylcellulose (Compound XXVI), to Polygeline (haemaccel) (Compound XXVII), to alginate (Compound LI), or to hydroxyethylstarch (Compound XXVIII), are useful for methods and in compositions as herein described but perform unexpectedly in terms of potency and range of useful pharmaceutical activity compared to the free conjugates. Thus, the combination of a phospholipid such as phosphatidylethanolamine, or related phospholipids which differ with regard to the polar head group, such as phosphatidylserine (PS), phosphatidylcholine (PC), phosphatidylinositol (PI), and phosphatidylglycerol (PG), results in the formation of a compound which has novel pharmacological properties when compared to the starting materials alone. In one embodiment, such properties may include: greater lubrication, greater local persistence, greater anti-inflammatory properties, greater antioxidant activity, or a combination thereof.

The biologically active compounds for use in the present invention or for incorporation in the lenses of the present invention described herein can have a wide range of molecular weights, e.g. above 50,000 (up to a few hundred thousands) when it is desirable to retain the lipid conjugate in the vascular system and below 50,000 when targeting to extravascular systems is desirable. The sole limitation on the molecular weight and the chemical structure of the conjugated moiety is that it does not result in a compound devoid of the desired biological activity, or lead to chemical or physiological instability to the extent that the Compound is rendered useless as a drug in the method of use described herein.

In one embodiment, the compounds for use in the present invention or for incorporation in the lenses of the present invention is represented by the structure of the general formula (A):

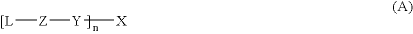

wherein

### L is a lipid or a phospholipid;

### Z is either nothing, ethanolamine, serine, inositol, choline, phosphate, or glycerol;

### Y is either nothing or a spacer group ranging in length from 2 to 30 atoms;

### X is a physiologically acceptable monomer, dimer, oligomer, or polymer; and

n is a number from 1 to 1000;
wherein any bond between L, Z, Y and X is either an amide or an esteric bond.

In one embodiment, the compounds for use in the present invention or for incorporation in the lenses of the present invention is represented by the structure of the general formula (A):

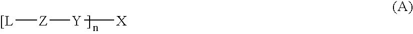

wherein

### L is a lipid or a phospholipid;

### Z is either nothing, ethanolamine, serine, inositol, choline, phosphate, or glycerol;

### Y is either nothing or a spacer group ranging in length from 2 to 30 atoms;

### X is a physiologically acceptable monomer, dimer, oligomer, or polymer; and

n is a number from 2 to 1000;
wherein any bond between L, Z, Y and X is either an amide or an esteric bond.

In one embodiment, L is phosphatidyl, Z is ethanolamine, wherein L and Z are chemically bonded resulting in phosphatidylethanolamine, Y is nothing, and X is carboxymethylcellulose. In another embodiment, L is phosphatidyl, Z is ethanolamine, wherein L and Z are chemically bonded resulting in phosphatidylethanolamine, Y is nothing, and X is a glycosaminoglycan. In one embodiment, the phosphatidylethanolamine moiety is dipalmitoyl phosphatidylethanolamine. In another embodiment, the phosphatidylethanolamine moiety is dimyristoyl phosphatidylethanolamine.

In another embodiment, the compounds for use in the present invention or for incorporation in the lenses of the present invention is represented by the structure of the general formula (I):

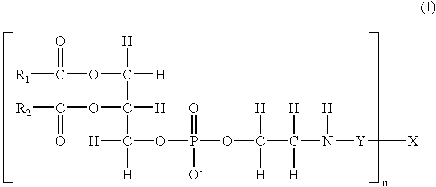

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Y is either nothing or a spacer group ranging in length from 2 to 30 atoms; and

### X is either a physiologically acceptable monomer, dimer, oligomer or a physiologically acceptable polymer; and

n is a number from 1 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

In another embodiment, the compounds for use in the present invention or for incorporation in the lenses of the present invention is represented by the structure of the general formula (I):

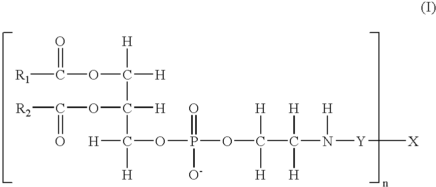

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Y is either nothing or a spacer group ranging in length from 2 to 30 atoms; and

### X is either a physiologically acceptable monomer, dimer, oligomer or a physiologically acceptable polymer; and

n is a number from 2 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

In one embodiment, the compounds for use in the present invention or for incorporation in the lenses of the present invention comprise one of the following as the conjugated moiety X: acetate, butyrate, glutarate, succinate, dodecanoate, didodecanoate, maltose, lactobionic acid, dextran, alginate, aspirin, cholate, cholesterylhemisuccinate, carboxymethyl-cellulose, heparin, hyaluronic acid, chondroitin sulfate, polygeline (haemaccel), hydroxyethylstarch (Hetastarch, HES) polyethyleneglycol, polycarboxylated polyethylene glycol, a glycosaminoglycan, a polysaccharide, a hetero-polysaccharide, a homo-polysaccharide, or a polypyranose. The polymers used as starting material to prepare the PE-conjugates may vary in molecular weight from 1 to 2,000 kDa.

Examples of phosphatidylethanolamine (PE) moieties are analogues of the phospholipid in which the chain length of the two fatty acid groups attached to the glycerol backbone of the phospholipid varies from 2-30 carbon atoms length, and in which these fatty acids chains contain saturated and/or unsaturated carbon atoms. In lieu of fatty acid chains, alkyl chains attached directly or via an ether linkage to the glycerol backbone of the phospholipid are included as analogues of PE. In one embodiment, the PE moiety is dipalmitoyl-phosphatidyl-ethanolamine.

In another embodiment, the PE moiety is dimyristoyl-phosphatidyl-ethanolamine.

Phosphatidyl-ethanolamine and its analogues may be from various sources, including natural, synthetic, and semi-synthetic derivatives and their isomers.

Phospholipids which can be employed in lieu of the PE moiety are N-methyl-PE derivatives and their analogues, linked through the amino group of the N-methyl-PE by a covalent bond; N,N-dimethyl-PE derivatives and their analogues linked through the amino group of the N,N-dimethyl-PE by a covalent bond, phosphatidylserine (PS) and its analogues, such as palmitoyl-stearoyl-PS, natural PS from various sources, semi-synthetic PSs, synthetic, natural and artifactual PSs and their isomers. Other phospholipids useful as conjugated moieties in this invention are phosphatidylcholine (PC), phosphafidylinositol (PI), phosphatidic acid and phosphoatidylglycerol (PG), as well as derivatives thereof comprising either phospholipids, lysophospholipids, phosphatidic acid, sphingomyelins, lysosphingomyelins, ceramide, and sphingosine.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group.

In another embodiment, the compounds for use in the present invention or for incorporation in the lenses of the present invention is represented by the structure of the general formula (II):

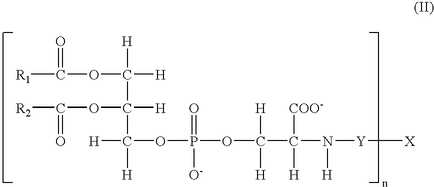

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Y is either nothing or a spacer group ranging in length from 2 to 30 atoms;

### X is a glycosaminoglycan; and

n is a number from 1 to 1000;
wherein if Y is nothing, the phosphatidylserine is directly linked to X via an amide bond and if Y is a spacer, the spacer is directly linked to X via an amide or an esteric bond and to the phosphatidylserine via an amide bond.

In another embodiment, the compounds for use in the present invention or for incorporation in the lenses of the present invention is represented by the structure of the general formula (II):

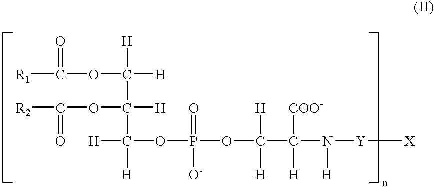

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Y is either nothing or a spacer group ranging in length from 2 to 30 atoms;

### X is a glycosaminoglycan; and

n is a number from 2 to 1000;
wherein if Y is nothing, the phosphatidylserine is directly linked to X via an amide bond and if Y is a spacer, the spacer is directly linked to X via an amide or an esteric bond and to the phosphatidylserine via an amide bond.

In one embodiment, the phosphatidylserine may be bonded to Y, or to X if Y is nothing, via the COO⁻ moiety of the phosphatidylserine.

In another embodiment, the compounds for use in the present invention or for incorporation in the lenses of the present invention is represented by the structure of the general formula (III):

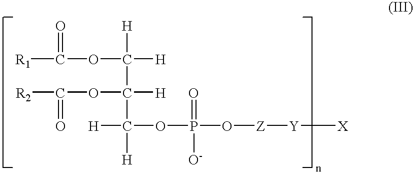

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 1 to 1000;
wherein any bond between the phosphatidyl, Z, Y and X is either an amide or an esteric bond.

In another embodiment, the compounds for use in the present invention or for incorporation in the lenses of the present invention is represented by the structure of the general formula (III):

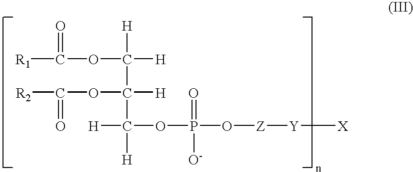

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 2 to 1000;
wherein any bond between the phosphatidyl, Z, Y and X is either an amide or an esteric bond.

In another embodiment, the compounds for use in the present invention or for incorporation in the lenses of the present invention is represented by the structure of the general formula (IV):

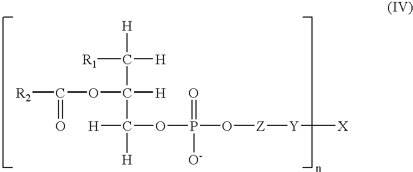

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IV):

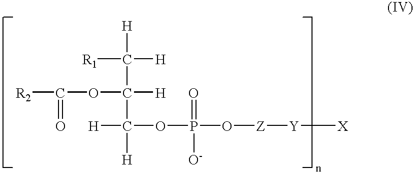

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (V):

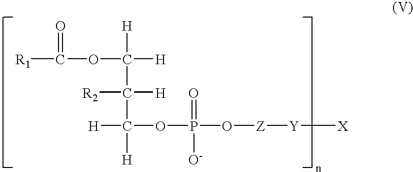

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (V):

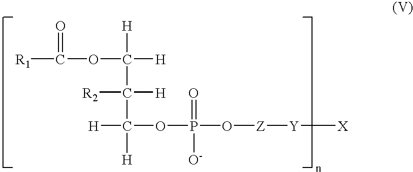

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (VI):

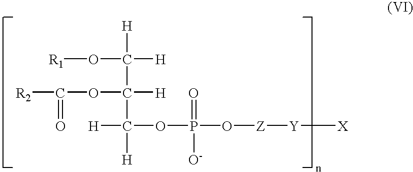

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention

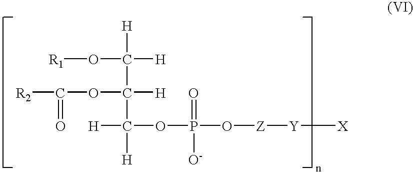

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, inositol, choline, or glycerol;

Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (VII):

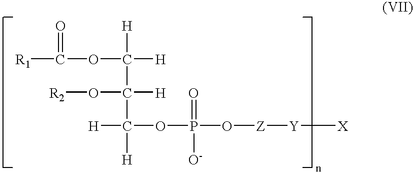

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (VII):

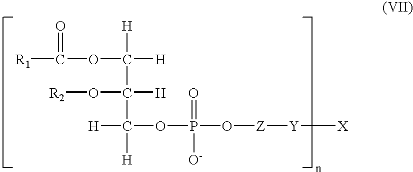

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In one embodiment of the invention, phosphatidylcholine (PC), phosphatidylinositol (PI), phosphatidic acid (PA), wherein Z is nothing, and phosphatidylglycerol (PG) conjugates are herein defined as compounds of the general formula (III).

In another embodiment, the compounds for use in the present invention or for incorporation in the lenses of the present invention is represented by the structure of the general formula (VIII):

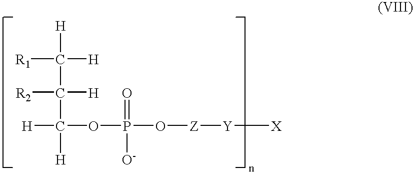

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (VIII):

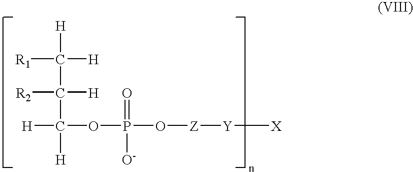

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IX):

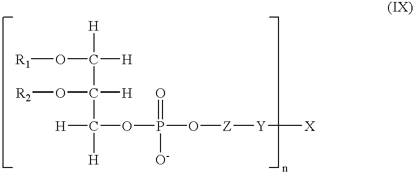

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IX):

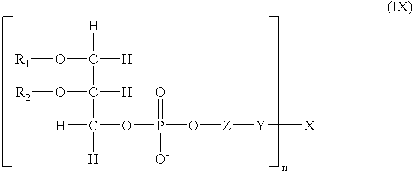

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IXa):

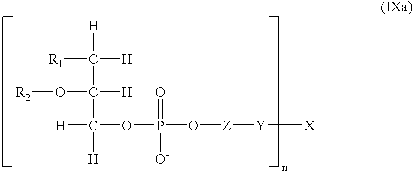

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IXa):

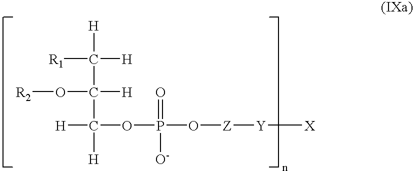

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IXb):

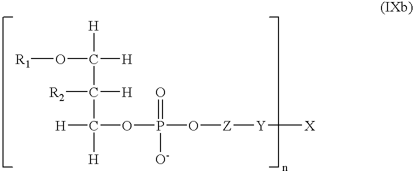

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the phospholipid, Z, Y and X is either an amide or an esteric bond.

In another embodiment, the compounds for use in the present invention or for incorporation in the lenses of the present invention is represented by the structure of the general formula (IXb):

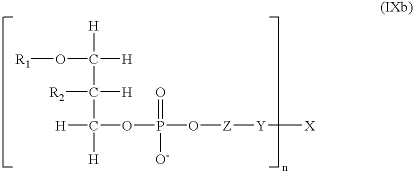

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 2 to 1000;
wherein any bond between the phospholipid, Z, Y and X is either an amide or an esteric bond.

In another embodiment, the compounds for use in the present invention or for incorporation in the lenses of the present invention is represented by the structure of the general formula (X):

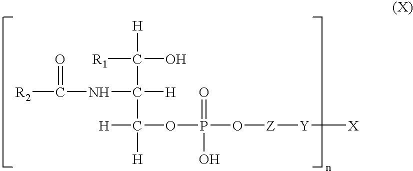

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the ceramide phosphoryl, Z, Y and X is either an amide or an esteric bond.

In another embodiment, the compounds for use in the present invention or for incorporation in the lenses of the present invention is represented by the structure of the general formula (X):

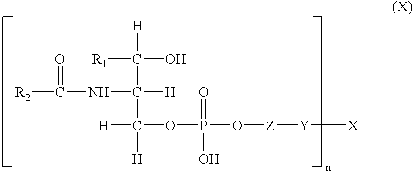

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, ethanolamine, serine, inositol, choline, or glycerol;

Y is either nothing or a spacer group ranging in length from 2 to 30 atoms;

### X is a glycosaminoglycan; and

n is a number from 2 to 1000;
wherein any bond between the ceramide phosphoryl, Z, Y and X is either an amide or an esteric bond.

In another embodiment, the compounds for use in the present invention or for incorporation in the lenses of the present invention is represented by the structure of the general formula (XI):

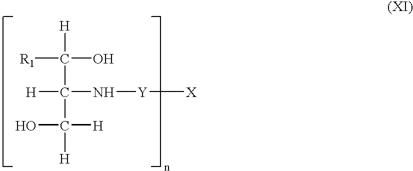

wherein

### R₁ is a linear, saturated, mono-unsaturated, or poly-unsaturated, alkyl chain ranging in length from 2 to 30 carbon atoms;

### Y is either nothing or a spacer group ranging in length from 2 to 30 atoms;

### X is a glycosaminoglycan; and

n is a number from 1 to 1000;
wherein if Y is nothing the sphingosyl is directly linked to X via an amide bond and if Y is a spacer, the spacer is directly linked to X and to the sphingosyl via an amide bond and to X via an amide or an esteric bond.

In another embodiment, the compounds for use in the present invention or for incorporation in the lenses of the present invention is represented by the structure of the general formula (XI):

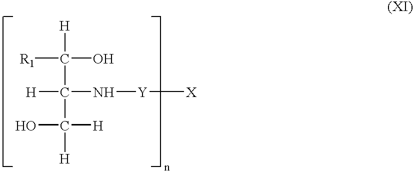

wherein

### R₁ is a linear, saturated, mono-unsaturated, or poly-unsaturated, alkyl chain ranging in length from 2 to 30 carbon atoms;

### Y is either nothing or a spacer group ranging in length from 2 to 30 atoms;

### X is a glycosaminoglycan; and

n is a number from 2 to 1000;
wherein if Y is nothing the sphingosyl is directly linked to X via an amide bond and if Y is a spacer, the spacer is directly linked to X and to the sphingosyl via an amide bond and to X via an amide or an esteric bond.

In another embodiment, the compounds for use in the present invention or for incorporation in the lenses of the present invention is represented by the structure of the general formula (XII):

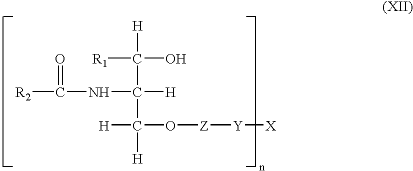

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 1 to 1000;
wherein any bond between the ceramide, Z, Y and X is either an amide or an esteric bond.

In another embodiment, the compounds for use in the present invention or for incorporation in the lenses of the present invention is represented by the structure of the general formula (XII):

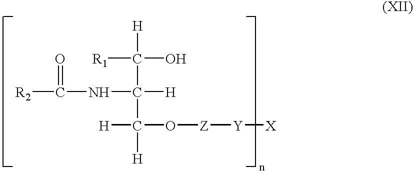

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, ethanolamine, serine, inositol, choline, or glycerol;

### Y is either nothing or a spacer group ranging in length from 2 to 30 atoms;

### X is a glycosaminoglycan; and

n is a number from 2 to 1000;
wherein any bond between the ceramide, Z, Y and X is either an amide or an esteric bond.

In another embodiment, the compounds for use in the present invention or for incorporation in the lenses of the present invention is represented by the structure of the general formula (XIII):

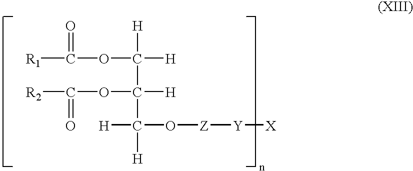

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the diglyceryl, Z, Y and X is either an amide or an esteric bond.

In another embodiment, the compounds for use in the present invention or for incorporation in the lenses of the present invention is represented by the structure of the general formula (XIII):

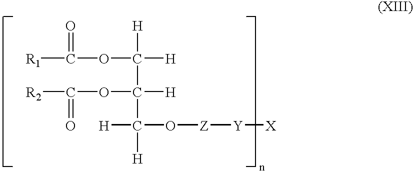

wherein

### R₁ is a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the diglyceryl, Z, Y and X is either an amide or an esteric bond.

In another embodiment, the compounds for use in the present invention or for incorporation in the lenses of the present invention is represented by the structure of the general formula (XIV):

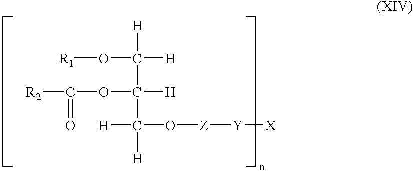

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the glycerolipid, Z, Y and X is either an amide or an esteric bond.

In another embodiment, the compounds for use in the present invention or for incorporation in the lenses of the present invention is represented by the structure of the general formula (XIV):

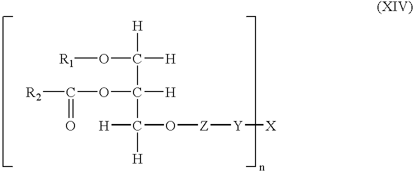

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the glycerolipid, Z, Y and X is either an amide or an esteric bond.

In another embodiment, the compounds for use in the present invention or for incorporation in the lenses of the present invention is represented by the structure of the general formula (XV):

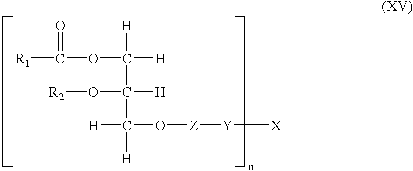

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the glycerolipid, Z, Y and X is either an amide or an esteric bond.

In another embodiment, the compounds for use in the present invention or for incorporation in the lenses of the present invention is represented by the structure of the general formula (XV):

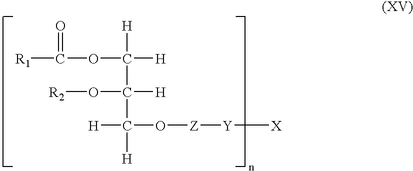

wherein

### R₁ is a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the glycerolipid, Z, Y and X is either an amide or an esteric bond.

In another embodiment, the compounds for use in the present invention or for incorporation in the lenses of the present invention is represented by the structure of the general formula (XVI):

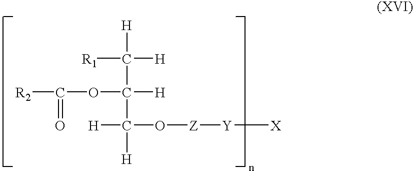

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XVI):

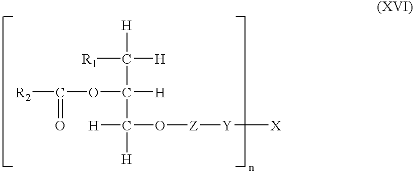

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

In another embodiment, the compounds for use in the present invention or for incorporation in the lenses of the present invention is represented by the structure of the general formula (XVII):

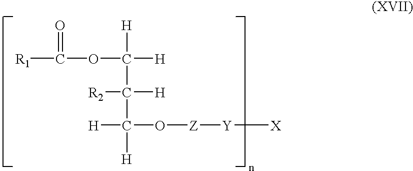

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XVII):

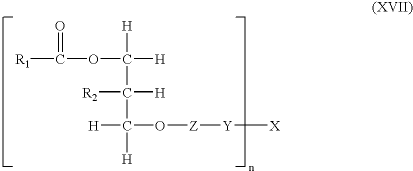

wherein

### R₁ is either hydrogen or a linear, saturated, mono-unsaturated, or poly-unsaturated, alkyl chain ranging in length from 2 to 30 carbon atoms;

### R₂ is a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

In another embodiment, the compounds for use in the present invention or for incorporation in the lenses of the present invention is represented by the structure of the general formula (XVIII):

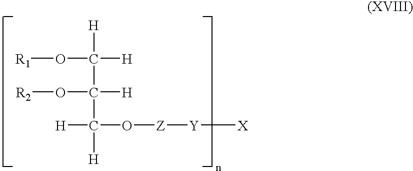

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XVIII):

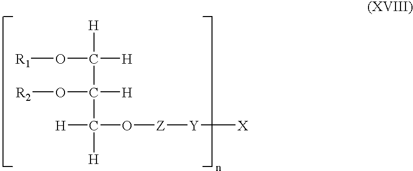

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

In another embodiment, the compounds for use in the present invention or for incorporation in the lenses of the present invention is represented by the structure of the general formula (XIX):

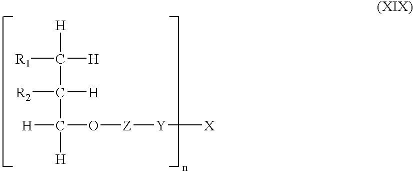

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XIX):

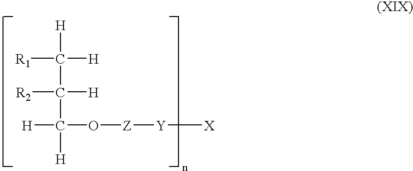

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

In another embodiment, the compounds for use in the present invention or for incorporation in the lenses of the present invention is represented by the structure of the general formula (XX):

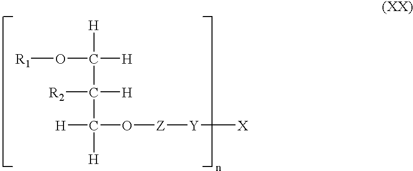

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XX):

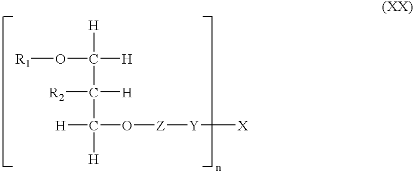

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

In another embodiment, the compounds for use in the present invention or for incorporation in the lenses of the present invention is represented by the structure of the general formula (XXI):

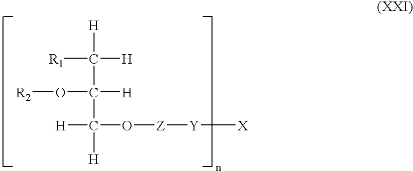

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 1 to 1000;
wherein any bond between the lipid, Z, Y and X is either an amide or an esteric bond.

In another embodiment, the compounds for use in the present invention or for incorporation in the lenses of the present invention is represented by the structure of the general formula (XXI):

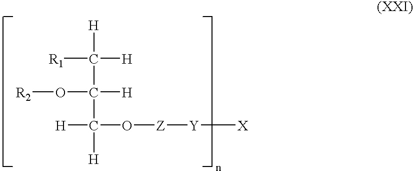

wherein

### R₁ is either hydrogen or a linear, saturated, mono-unsaturated, or poly-unsaturated, alkyl chain ranging in length from 2 to 30 carbon atoms;

### R₂ is either hydrogen or a linear, saturated, mono-unsaturated, or poly-unsaturated, alkyl chain ranging in length from 2 to 30 carbon atoms;

### Z is either nothing, choline, phosphate, inositol, or glycerol;

### Y is either nothing or a spacer group ranging in length from 2 to 30 atoms;

### X is a glycosaminoglycan; and

n is a number from 2 to 1000;
wherein any bond between the lipid, Z, Y and X is either an amide or an esteric bond.

For any or all of the compounds represented by the structures of the general formulae (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), and (XXII) hereinabove: In one embodiment, X is a glycosaminoglycan. According to this aspect and in one embodiment, the glycosaminoglycan may be, inter alia, hyaluronic acid, heparin, heparan sulfate, chondroitin sulfate, keratin, keratan sulfate, dermatan sulfate or a derivative thereof. In another embodiment, X is not a glycosaminoglycan. In another embodiment, X is a polysaccharide, which in one embodiment is a hetero-polysaccharide, and in another embodiment, is a homo-polysaccharide. In another embodiment, X is a polypyranose.

In another embodiment, the glycosaminoglycan is a polymer of disaccharide units. In another embodiment, the number of the disaccharide units in the polymer is m. In another embodiment, m is a number from 2-10,000. In another embodiment, m is a number from 2-500. In another embodiment, m is a number from 2-1000. In another embodiment, m is a number from 50-500. In another embodiment, m is a number from 2-2000. In another embodiment, m is a number from 500-2000. In another embodiment, m is a number from 1000-2000. In another embodiment, m is a number from 2000-5000. In another embodiment, m is a number from 3000-7000. In another embodiment, m is a number from 5000-10,000. In another embodiment, a disaccharide unit of a glycosaminoglycan may be bound to one lipid or phospholipid moiety. In another embodiment, each disaccharide unit of the glycosaminoglycan may be bound to zero or one lipid or phospholipid moieties. In another embodiment, the lipid or phospholipid moieties are bound to the —COOH group of the disaccharide unit. In another embodiment, the bond between the lipid or phospholipid moiety and the disaccharide unit is an amide bond.

In one embodiment, the compounds for use in the present invention or for incorporation in the lenses of the present invention comprises a glycosaminoglycan (GAG), and further comprises total phospholipid such that the molar ratio between the phospholipid and the GAG is in the range of between 1.5:1 to 20:1. In another embodiment, the molar ratio between the phospholipid and the GAG is in the range of between 1.5:1 to 10:1. In another embodiment, the molar ratio between the phospholipid and the GAG is in the range of between 1.5:1 to 5:1. In another embodiment, the molar ration between the phospholipid and the GAG is 1.5:1. In another embodiment, the molar ration between the phospholipid and the GAG is 2:1. In another embodiment, the molar ration between the phospholipid and the GAG is 3:1. In another embodiment, the molar ration between the phospholipid and the GAG is 5:1. In another embodiment, the molar ration between the phospholipid and the GAG is 10:1. In another embodiment, the molar ration between the phospholipid and the GAG is 20:1.

In one embodiment, the compounds for use in the present invention or for incorporation in the lenses of the present invention comprises a glycosaminoglycan (GAG) with a molecular weight in the range of between 30-100 kD. In another embodiment, the GAG has a molecular weight in the range of between 30-80 kD. In another embodiment, the GAG has a molecular weight in the range of between 30-50 kD. In another embodiment, the GAG has a molecular weight in the range of between 20-80 kD. In another embodiment, the GAG has a molecular weight in the range of between 20-50 kD.

In another embodiment, the chondroitin sulfate may be, inter alia, chondroitin-6-sulfate, chondroitin-4-sulfate or a derivative thereof.

In one embodiment of the invention, Y is nothing. Non-limiting examples of suitable divalent groups forming the optional bridging group (which in one embodiment, is referred to as a spacer) Y, according to embodiments of the invention, are straight or branched chain alkylene, e.g. of 2 or more, preferably 4 to 30 carbon atoms, —CO-alkylene-CO, —NH-alkylene-NH—, —CO—alkylene-NH—, —NH-alkylene-NH, CO-alkylene-NH—, an amino acid, cycloalkylene, wherein alkylene in each instance, is straight or branched chain and contains 2 or more, preferably 2 to 30 atoms in the chain, —(—O—CH(CH₃)CH₂—)ₓ—wherein x is an integer of 1 or more.

According to embodiments of the invention, in addition to the traditional phospholipid structure, related derivatives for use in this invention are phospholipids modified at the C1 or C2 position to contain an amine, ether or alkyl bond instead of an ester bond. In one embodiment of the invention, the alkyl phospholipid derivatives and ether phospholipid derivatives are exemplified herein.

In one embodiment of the invention, the sugar rings of the glycosaminoglycan are intact. In another embodiment, intact refers to closed. In another embodiment, intact refers to natural. In another embodiment, intact refers to unbroken.

In one embodiment of the invention, the structure of the lipid or phospholipid in the compounds for use in the present invention or for incorporation in the lenses of the present invention is intact. In another embodiment, the natural structure of the lipid or phospholipids in the compounds for use in the present invention or for incorporation in the lenses of the present invention is maintained.

In one embodiment, the compounds for use in the present invention or for incorporation in the lenses of the present invention are biodegradable.

In one embodiment, the compounds for use in the present invention or for incorporation in the lenses of the present invention is phosphatidylethanolamine bound to aspirin. In one embodiment, the compound according to the invention is phosphatidylethanolamine bound to glutarate.

In some embodiments, the compounds for use in the present invention or for incorporation in the lenses of the present invention are as listed in Table 1 below.

TABLE 1

| Phospholipid | | | |
|---|---|---|---|
| Compound | Spacer | Polymer (m.w.) | |
| PE | None | Hyaluronic acid | XXII |
| | | (2-2000 kDa) | |
| Dimyristoyl- | None | Hyaluronic acid | XXIII |
| PE | | | |
| PE | None | Heparin | XXIV |
| | | (0.5-110 kDa) | |
| PE | None | Chondroitin sulfate A | XXV |
| PE | None | Carboxymethylcellulose | XXVI |
| | | (20-500 kDa) | |
| PE | Dicarboxylic | Polygeline | XXVII |
| | acid + Diamine | (haemaccel) | |
| | | (4-40 kDa) | |
| PE | None | Hydroxyethylstarch | XXVIII |
| PE | Dicarboxylic | Dextran | XXIX |
| | acid + Diamine | (1-2,000 kDa) | |
| PE | None | Aspirin | XXX |
| PE | Carboxyl | Hyaluronic acid | XXXI |
| | amino | (2-2000 kDa) | |
| | group | | |
| PE | Dicarboxyl | Hyaluronic acid | XXXII |
| | group | (2-2000 kDa) | |
| PE | Dipalmitoic | Hyaluronic acid | XXXIII |
| | acid | (2-2000 kDa) | |
| PE | Carboxyl | Heparin | XXXIV |
| | amino | (0.5-110 kDa) | |
| | group | | |
| PE | Dicarboxyl | Heparin | XXXV |
| | group | (0.5-110 kDa) | |
| PE | Carboxyl | Chondroitin sulfate A | XXXVI |
| | amino | | |
| | group | | |
| PE | Dicarboxyl | Chondroitin sulfate A | XXXVII |
| | group | | |
| PE | Carboxyl | Carboxymethylcellulose | XXXVIII |
| | amino | (20-500 kDa) | |
| | group | | |
| PE | Dicarboxyl | Carboxymethylcellulose | XXXIX |
| | group | (20-500 kDa) | |
| PE | None | Polygeline | XL |
| | | (haemaccel) | |
| | | (4-40 kDa) | |
| PE | Carboxyl | Polygeline (haemaccel) | XLI |
| | amino | (4-40 kDa) | |
| | group | | |
| PE | Dicarboxyl | Polygeline (haemaccel) | XLII |
| | group | (4-40 kDa) | |
| PE | Carboxyl | Hydroxyethylstarch | XLIII |
| | amino | | |
| | group | | |
| PE | Dicarboxyl | Hydroxyethylstarch | XLIV |
| | group | | |
| PE | None | Dextran | XLV |
| | | (1-2,000 kDa) | |
| PE | Carboxyl | Dextran | XLVI |
| | amino | (1-2,000 kDa) | |
| | group | | |
| PE | Dicarboxyl | Dextran | XLVII |
| | group | (1-2,000 kDa) | |
| PE | Carboxyl | Aspirin | XLVIII |
| | amino | | |
| | group | | |
| PE | Dicarboxyl | Aspirin | XLIX |
| | group | | |
| PE | None | Albumin | L |
| PE | None | Alginate | LI |
| | | (2-2000 kDa) | |
| PE | None | Polyaminoacid | LII |
| PE | None | Polyethylene glycol | LIII |
| PE | None | Lactobionic acid | LIV |
| PE | None | Acetylsalicylate | LV |
| PE | None | Cholesteryl- | LVI |
| | | hemmisuccinate | |
| PE | None | Maltose | LVII |
| PE | None | Cholic acid | LVIII |
| PE | None | Chondroitin sulfates | LIX |
| PE | None | Polycarboxylated | LX |
| | | polyethylene glycol | |
| Dipalmitoyl- | None | Hyaluronic acid | LXI |
| PE | | | |
| Dipalmitoyl- | None | Heparin | LXII |
| PE | | | |
| Dipalmitoyl- | None | Chondroitin sulfate A | LXIII |
| PE | | | |
| Dipalmitoyl- | None | Carboxymethylcellulose | LXIV |
| PE | | | |
| Dipalmitoyl- | None | Polygeline (haemaccel) | LXV |
| PE | | | |
| Dipalmitoyl- | None | Hydroxyethylstarch | LXVI |
| PE | | | |
| Dipalmitoyl- | None | Dextran | LXVII |
| PE | | | |
| Dipalmitoyl- | None | Aspirin | LXVIII |
| PE | | | |
| Dimyristoyl- | None | Heparin | LXVIX |
| PE | | | |
| Dimyristoyl- | None | Chondroitin sulfate A | LXX |
| PE | | | |
| Dimyristoyl- | None | Carboxymethylcellulose | LXXI |
| PE | | | |
| Dimyristoyl- | None | Polygeline (haemaccel) | LXXII |
| PE | | | |
| Dimyristoyl- | None | Hydroxyethylstarch | LXXIII |
| PE | | | |
| Dimyristoyl- | None | Dextran | LXXIV |
| PE | | | |
| Dimyristoyl- | None | Aspirin | LXXV |
| PE | | | |
| PS | None | Hyaluronic acid | LXXVI |
| PS | None | Heparin | LXXVII |
| PS | None | Polygeline (haemaccel) | LXXVIII |
| PC | None | Hyaluronic acid | LXXIX |
| PC | None | Heparin | LXXX |
| PC | None | Polygeline (haemaccel) | LXXXI |
| PI | None | Hyaluronic acid | LXXXII |
| PI | None | Heparin | LXXXIII |
| PI | None | Polygeline (haemaccel) | LXXXIV |
| PG | None | Hyaluronic acid | LXXXV |
| PG | None | Heparin | LXXXVI |
| PG | None | Polygeline (haemaccel) | LXXXVII |
| PE | None | Glutaryl | LXXXVIII |
| Dipalmitoyl- | None | Alginate | LXXXIX |
| PE | | | |
| Dimyristoyl- | None | Alginate | XC |
| PE | | | |
| PS | None | Alginate | XCI |
| PC | None | Alginate | XCII |
| PI | None | Alginate | XCIII |
| PG | None | Alginate | XCIV |
| PS | None | Hydroxyethylstarch | XCV |
| PC | None | Hydroxyethylstarch | XCVI |
| PI | None | Hydroxyethylstarch | XCVII |
| PG | None | Hydroxyethylstarch | XCVIII |
| PE | —CO—(CH₂)₃—CO—NH—(CH₂)₆— | Hydroxyethylstarch | XCIX |
| PE | —CO—CH₂— | Carboxymethylcellulose | C |

In one embodiment of the invention, the compounds for use in the present invention or for incorporation in the lenses of the present invention are any one or more of Compounds I-C. In another embodiment, the invention provides a contact lens comprising any combination of any of the compounds of the invention or the use of any combination of any of the compounds as herein described. In another embodiment, the invention provides a contact lens comprising Compounds XCIX, C, or a combination thereof and uses thereof. In another embodiment, the invention provides a contact lens comprising Compounds LXV, LXVI, LXXI, LXXII, LXXIII, LXXXIX, XC, or a combination thereof and uses thereof. In another embodiment, the compounds for use in the present invention or incorporated in the contact lenses of this invention are Compound XXII, Compound XXIII, Compound XXIV, Compound XXV, Compound XXVI, Compound XXVII, Compound XXVIII, Compound XXIX, Compound XXX, Compound LI, or pharmaceutically acceptable salts thereof, in combination with a physiologically acceptable carrier or solvent. According to embodiments of the invention, these polymers, when chosen as the conjugated moiety, may vary in molecular weights from 200 to 2,000,000 Daltons. In one embodiment of the invention, the molecular weight of the polymer as referred to herein is from 200 to 1000 Daltons. In another embodiment, the molecular weight of the polymer as referred to herein is from 200 to 1000 Daltons. In another embodiment, the molecular weight of the polymer as referred to herein is from 1000 to 5000 Daltons. In another embodiment, the molecular weight of the polymer as referred to herein is from 5000 to 10,000 Daltons. In another embodiment, the molecular weight of the polymer as referred to herein is from 10,000 to 20,000 Daltons. In another embodiment, the molecular weight of the polymer as referred to herein is from 10,000 to 50,000 Daltons. In another embodiment, the molecular weight of the polymer as referred to herein is from 20,000 to 70,000 Daltons. In another embodiment, the molecular weight of the polymer as referred to herein is from 50,000 to 100,000 Daltons. In another embodiment, the molecular weight of the polymer as referred to herein is from 100,000 to 200,000 Daltons. In another embodiment, the molecular weight of the polymer as referred to herein is from 200,000 to 500,000 Daltons. In another embodiment, the molecular weight of the polymer as referred to herein is from 200,000 to 1,000,000 Daltons. In another embodiment, the molecular weight of the polymer as referred to herein is from 500,000 to 1,000,000 Daltons. In another embodiment, the molecular weight of the polymer as referred to herein is from 1,000,000 to 2,000,000 Daltons. Various molecular weight species have been shown to have the desired biological efficacy. In one embodiment, AlgPE has a molecular weight of approximately 120 kD, CSAPE has a molecular weight of approximately 100 kD, HemPE has a molecular weight of approximately 75 kD, HesDMPE has a molecular weight of approximately 90 kD, CMPE has a molecular weight of approximately 75 kD, or a combination thereof. In one embodiment, “approximately” refers to up to 5%, 10%, 15%, 20%, or 25% of the value. In another embodiment, “approximately” refers to 5-25%, 5-15%, 10-25%, 10-20%, 15-25% of the value.

In one embodiment of this invention, low molecular weight compounds for use in the present invention or incorporated in the contact lenses of this invention are defined hereinabove as the compounds of formula (I)-(XXI) wherein X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid, glycosaminoglycan, or polypyranose.

Examples of suitable divalent groups forming the optional bridging group Y are straight- or branched -chain alkylene, e.g. of 2 or more, preferably 4 to 18 carbon atoms, —CO-alkylene-CO, —NH-alkylene-NH—, —CO—alkylene-NH—, cycloalkylene, wherein alkylene in each instance, is straight or branched chain and contains 2 or more, preferably 2 to 18 carbon atoms in the chain, —(—O—CH(CH₃)CH₂—)ₓ— wherein x is an integer of 1 or more.

In another embodiment, in addition to the traditional phospholipid structure, related derivatives for use in this invention are phospholipids modified at the C1 or C2 position to contain an ether or alkyl bond instead of an ester bond. These derivatives are exemplified hereinabove by the general formulae (VIII) and (IX).

In one embodiment of the invention, X is covalently conjugated to a lipid. In another embodiment, X is covalently conjugated to a lipid via an amide bond. In another embodiment, X is covalently conjugated to a lipid via an esteric bond. In another embodiment, the lipid is phosphatidylethanolamine.

In one embodiment, cell surface GAGs play a key role in protecting cells from diverse damaging agents and processes, such as reactive oxygen species and free radicals, endotoxins, cytokines, invasion promoting enzymes, and agents that induce and/or facilitate degradation of extracellular matrix and basal membrane, cell invasiveness, white cell extravasation and infiltration, chemotaxis, and others. In addition, cell surface GAGs protect cells from bacterial, viral and parasitic infection and their stripping exposes the cell to interaction and subsequent internalization of the microorganism. Enrichment of cell surface GAGs would thus assist in protection of the cell from injurious processes. Thus, in one embodiment of the invention, PLA2 inhibitors are conjugated to GAGs or GAG-mimicking molecules. In another embodiment, these compounds for use in the present invention or incorporated in the contact lenses of this invention provide wide-range protection from diverse injurious processes, and are effective in amelioration of diseases that requires cell protection from injurious biochemical mediators.

In another embodiment, a GAG-mimicking molecule may be, inter alia, a negatively charged molecule. In another embodiment, a GAG-mimicking molecule may be, inter alia, a salicylate derivative. In another embodiment, a GAG-mimicking molecule may be, inter alia, a dicarboxylic acid.

In another embodiment, the invention provides a contact lens for treating a subject suffering from an eye disease, including a lipid or phospholipid moiety bonded to a physiologically acceptable monomer, dimer, oligomer, or polymer; and a pharmaceutically acceptable carrier or excipient.

In another embodiment, the invention provides a contact lens for treating a subject suffering from an eye disease, including any one of the compounds for use in the present invention or any combination thereof; and a pharmaceutically acceptable carrier or excipient. In another embodiment, the compounds for use in the present invention include, inter alia, the compounds represented by the structures of the general formulae as described hereinbelow: (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII), or any combination thereof.

The combination of lipids, such as, but not limited to phosphatidylethanolamine and phosphatidylserine, with additional monomer or polymer moieties, is thus a practical route to the production of new drugs for medical purposes, provided that the resultant chemical composition displays the desired range of pharmacological properties. In one embodiment, the compounds for use in the present invention possess a combination of multiple and potent pharmacological effects in addition to the ability to inhibit the extracellular form of the enzyme phospholipase A2. While the pharmacological activity of the compounds for use in the present invention described herein may be due in part to the nature of the lipid moiety, the multiple and diverse combination of pharmacological properties observed for the compounds for use in the present invention emerges from the ability of the compound structure to act essentially as several different drugs in one chemical entity.

In the cases described herein, the diversity of biological activities and the effectiveness in disease exhibited by the compounds for use in the present invention or incorporated in the contact lenses of this invention far exceed the properties anticipated by use of the starting materials themselves, when administered alone or in combination. However, the phospholipid conjugate compounds, alone or in combination, are valuable when used in the methods of treating diseases and conditions specifically described herein.

### Eve Devices

It is to be understood that the compounds for use in the present invention may also be used in combination with any device which is applied to an eye surface or applied to the internal regions of the eye. In one embodiment, such a device is a contact lens, while in other embodiments, it is a corneal prosthetic device, prosthetic iris implant, scleral lens prosthetic device, an intra-ocular implant, a scieral buckle, ophthalmic tantalum clip, opthalmic conformer, artificial eye, absorbable implant, eye sphere implant, extraocular orbital implant, keratoprosthesis, intraocular lens, scleral shell, eye valve implant, or a combination thereof.

In one embodiment, the present invention provides a substrate having a coating on at least a portion of a surface of said substrate, said coating comprising a lipid or phospholipid moiety bound to a physiologically acceptable monomer, dimer, oligomer, or polymer as any of the embodiments describe hereinabove. In one embodiment, the physiological acceptable monomer, dimer, oligomer, or polymer is a polypyranose. In one embodiment, the substrate is a contact lens. In one embodiment, the substrate is an implant. In one embodiment, the substrate is part of a device for ophthalmic or opthamologic use.

In one embodiment, the present invention provides a contact lens having a coating on at least a portion of a surface of said contact lens, said coating comprising a lipid or phospholipid moiety bound to a physiologically acceptable monomer, dimer, oligomer, or polymer. In one embodiment, the coating comprises a lipid or phospholipid moiety bound to a polypyranose, or, in another embodiment, a lipid or phospholipid moiety bound to a hetero- or homo-polysaccharide, or combination thereof. In one embodiment, the present invention provides a method of protecting a contact lens from accumulating protein deposits, infectious agents, or a combination thereof.

It is to be understood that a contact lens or other substrate comprising a compound of the instant invention as used herein, refers to a contact lens or other substrate for which the compound coats, partially coats, embeds within, adsorbs to, absorbs within, or penetrates a contact lens.

In one embodiment, the coating is prepared and applied as an aqueous solution, a suspension, or a colloid and then applied to the substrate according to any process that will put the coating in uniform contact with the substrate, which in one embodiment is immersion, spraying, brushing, spin coating, or a combination thereof. In one embodiment, immersion and spraying are the preferred processes since proper thickness and uniformity of the coating are most easily accomplished with them. In another embodiment, coating thickness at the submicron level is achieved by preparing a dilute polymer solution of coating material such as a solution between about 0.1 and 6.0 wt %. In one embodiment, a silicon-based polymer substrate is then immersed in the polymer solution of coating material for between 1 min and 120 minutes at temperatures of 5-80° C. followed by a five step rinse over approximately 30 min in which the unreacted polymer is washed away using a buffered saline solution. Other methods of coating intra-ocular devices that are known in the art may be performed as well. In another embodiment, compounds for use in the present invention are applied via soaking a substrate, including inter alia, a contact lens in a solution including, inter alia, a contact lens solution.

In another embodiment, the association of the compound and the contact lens may be by any means known in the Art. In one embodiment, the compound integrates into the internal structure of the lens, while in another embodiment, the compound remains on the surface. In another embodiment, the compound penetrates the lens to some degree, with a depth ranging from 5-95%. In some embodiments, the depth is 5%, 10%, 20%, 40%, 50%, 70%, 90%, or 95%. In one embodiment, the compound is dispersed uniformly throughout the lattice of the lens, while in another embodiment, the compound is concentrated in a particular region of the lens. In one embodiment, the compound is used to coat the contact lens on at least a portion of its surface, which in one embodiment is the surface adjacent to the cornea, exterior surface, both surfaces or portions thereof. In another embodiment, the compound is fully or partially embedded within the contact lens. In another embodiment, the compound is both on the lens surface and embedded within the lens. In one embodiment, the compounds for use in the instant invention embed by intercalating into voids in the contact lens matrix.

In one embodiment, contact lenses for the purposes of this invention have defined surfaces with a particular chemistry such that compounds can be readily adsorbed thereonto. In another embodiment, the lens may comprise a material that promotes the adhesion of the compounds to the lens. In another embodiment, the compound may be capable of adhering directly to the lens or other substrate. In another embodiment, the compounds of the instant invention may be used as a method of adhering other compounds to a surface, which is one embodiment is adhering pigments to the surface of a contact lens. In one embodiment, adherence is via chemical bonding, which in one embodiment is covalent, ionic, hydrophilic, hydrogen bonds, van der Waals forces, or a combination thereof. In one embodiment, the compound must be capable of remaining affixed to the lens substrate throughout the useful lifespan of the lens (storage time plus the time in which it will be in contact with a user's eye). In one embodiment, the lens is coated with one or more layers of a coating material. In one embodiment, a tielayer or coupling agent can be used to adhere a hydrophilic coating to a lens or other substrate. A coating layer with a significant number of carbonyl groups could be bonded to a polyolefin substrate through the use of a diamine tielayer in such a scheme. Selections of compatible lens substrate, coating, and tielayer (if necessary) materials is well within the knowledge of one skilled in the art. In one embodiment, the compounds of the present invention are used together with other coating compounds known in the Art.

In one embodiment, contact lenses may be soft contact lenses, while in one embodiment, they may be hard contact lenses, made of materials known in the Art. Lens forming materials that are suitable in the fabrication of contact lenses are illustrated by one or more of the following U.S. Pat. Nos. 2,976,576; 3,220,960; 3,937,680; 3,948,871; 3,949,021; 3,983,083; 3,988,274; 4,018,853; 3,875,211; 3,503,942; 3,532,679; 3,621,079; 3,639,524; 3,700,761; 3,721,657; 3,758,448; 3,772,235; 3,786,034; 3,803,093; 3,816,571; 3,940,207; 3,431,046; 3,542,461; 4,055,378; 4,064,086; 4,062,624; and 5,034,166. In one embodiment, the compounds are introduced into the structure of the lens material during the manufacturing process. In one embodiment, the contact lens is a daily-wear lens, a continuous-wear lens or has properties of both. In one embodiment, the contact lens is made from silicone-containing materials. In one embodiment, the contact lens material is a hydrogel and in another embodiment, a non-hydrogel. In one embodiment, fluorine-containing groups are introduced into contact lens polymers, which in one embodiment increase oxygen permeability, as is known to one of skill in the Art.

“Contact Lens” refers to a structure that can be placed on or within a wearer's eye. A contact lens can correct, improve, or alter a user's eyesight, but that need not be the case. A contact lens can be of any appropriate material known in the art or later developed, and can be a soft lens, a hard lens or a hybrid lens. A contact lens can be in a dry state or a wet state. In one embodiment, a contact lens may be a planned replacement contact lens or a disposable contact lens.

“Soft Lens” refers to a variety of soft lenses as they are known in the art that are characterized as having, for example, at least one of the following characteristics: oxygen permeable, hydrophilic or pliable. “Hard Lens” refers to a variety of hard lenses as they are known in the art that are characterized as having, for example, at least one of the following characteristics: hydrophobic, gas permeable or rigid. “Hybrid Lens” refers to a variety of hybrid lenses as they are known in the art, such as, for example, a lens having a soft skirt and a hard center.

“Dry State” refers to a soft lens in a state prior to hydration or the state of a hard lens under storage or use conditions. “Wet State” refers to a soft lens in a hydrated state.

In another embodiment, a device for use with contact lenses may comprise compounds for use in the instant invention. In one embodiment, such a device may be a standard device commercially available for the storage, cleaning, disinfection, and/or carriage of contact lenses, which in one embodiment, is characterized by a hollow well. In one embodiment, a single device may be suitable for storage, cleaning, disinfection, and/or carriage of contact lenses, while in another embodiment, each is a separate device.

In one embodiment, the substrate, which in one embodiment is a contact lens, contact lens device, contact lens solution and/or intraocular device, comprising compounds for use in the instant invention suppresses, inhibits, prevents or treats eye-related disorders, including inter alia, those described hereinabove, in a subject. In another embodiment, the substrate comprising compounds for use in the instant invention prevents or treats proteinaceous deposits accumulating on the substrate. In another embodiment, the compounds for use in the instant invention endows the surface of the substrate with the property of being more hydrophilic, which in one embodiment, may increase comfort, decrease eye dryness, or a combination thereof. In another embodiment, the compounds for use in the instant invention prevent adverse reactions that are directly or indirectly related to the substrate, such as corneal edema, inflammation, or lymphocyte infiltration. In another embodiment, the substrate comprising compounds for use in the instant invention increases wettability, decreases adhesion, increases biocompatability, provides UV shielding, prevents glare, decreases dryness, grittiness, general discomfort, prevents microbial (in one embodiment, bacterial) infections, or a combination thereof or provides other desirable characteristics and properties to the substrate that are known in the Art.

In some embodiments, the contact lenses of this invention are useful in treating ocular surface disease in a subject, via applying a lens of claim 1 to the eye of a subject. In some embodiments, the disease is dry eye or Keratoconus.

Some causes of dry eyes are an underlying medical condition, such as Sjogren's syndrome, graft versus host disease (following bone marrow transplants), radiation treatment in the eye area, Stevens-Johnson syndrome, and autoimmune disorders. In some cases the cause is unknown (idiopathic). The condition can be due to a diminished supply of tears, excessively rapid evaporation or both. Dry eyes is one of the most common ocular complaints. Symptoms vary from mildly annoying, intermittent dryness and burning and paradoxical tearing (worse in dry environments) to constant disabling pain, increased light sensitivity and blurred vision. Mild to moderate cases of dry eyes can usually be controlled by the frequent use of artificial tears, punctal plugs that slow the drainage of tears from the eyes and a new, prescription eye drop (Restasis) that may reduce the inflammation associated with dry eyes. This group constitutes the vast majority of dry eyes. However, it is the patient with severe, disabling dry eyes unresponsive to the above treatments who can benefit the most from the lenses of this invention.

Keratoconus is another eye condition can be addressed by the use of a lens of this invention. Keratoconus is characterized by a progressive thinning and steepening of the central cornea. As the cornea steepens and thins, a patient experiences a decrease in vision which can be mild or severe depending on the severity of the disease. Keratoconus has no known cure, thus the lenses of this invention offer a means to address this disease.

In some embodiments, the invention is directed to treating cataracts, whereby a lens of a subject is removed, treated with the compounds of this invention and reinserted in the subject, thereby treating the cataracts, wherein the compounds apply improve lens function in the subject.

Considering the possibilities of irritation and damage to the cornea, the stratum, the endothelium or other parts of the eyes are manifold, one requires protection, conditioning and whenever possible, restoration of the sustained damage to the eye. The current invention therefore provides contact lenses, a method for the manufacture of contact lenses and use therefore in the treatment and/or care and/or protection of the eyes, comprising the incorporation of the compounds as herein described within contact lenses for the treatment and/or care and/or protection of the eyes. By wearing contact lenses the compounds as herein described, which treat and/or protect and/or care of the eyes will be in contact with (part of) the eyes. This way the lens will be a method of administering these compounds which will often imply a more long-lasting administering compared to current eye-drops. The compounds' mentioned can be either absorbed into the lens material or be attached to them or both.

The lens may be useful in treating retinal disease, or in ameliorating conditions which arise as a result of such treatment, for example as described in U.S. Pat. No. 5,719,656, fully incorporated by reference herein, where a lens has the characteristics described therein, and incorporates the compounds herein described.

### Preparation of Compounds for Use in the Present Invention

In one embodiment, the preparation of high molecular weight compounds for use in the methods of the present invention is as described in U.S. Pat. No. 5,064,817, which is incorporated fully herein by reference. In one embodiment, these synthetic methods are applicable to the preparation of low molecular weight compounds for use in the present invention as well, i.e. compounds for use in the present invention comprising monomers and dimers as the conjugated moiety, with appropriate modifications in the procedure as would be readily evident to one skilled in the art. The preparation of some low molecular weight compounds for use in the present invention may be conducted using methods well known in the art or as described in U.S. patent application Ser. No. 10/952,496, which is incorporated herein by reference in its entirety. Without further elaboration, it is believed that one skilled in the art can, using the preceding description, utilize the present invention to its fullest extent.

It will be appreciated by persons skilled in the art that the present invention is not limited by what has been particularly shown and described herein above and that numerous modifications, all of which fall within the scope of the present invention, exist. Rather, the scope of the invention is defined by the claims which follow:

What we claim is:

1. A contact lens comprising a lipid or phospholipid moiety bound optionally via a spacer to a physiologically acceptable monomer, dimer, oligomer, or polymer via an ester or amide bond, and/or a pharmaceutically acceptable salt or a pharmaceutical product thereof.

2. The lens of claim 1, wherein said phospholipid moiety is phosphatidylethanolamine.

3. The lens of claim 2, wherein said phosphatidylethanolamine is dipalmitoyl phosphatidylethanolamine.

4. The lens of claim 3, wherein said phosphatidylethanolamine is dimyristoyl phosphatidylethanolamine.

5. The lens of claim 1, wherein said physiologically acceptable monomer, dimer, oligomer, or polymer is polygeline.

6. The lens of claim 1, wherein said physiologically acceptable monomer, dimer, oligomer, or polymer is a polypyranose.

7. The lens of claim 6, wherein said polypyranose is carboxymethylcellulose.

8. The lens of claim 6, wherein said polypyranose is alginate.

9. The lens of claim 6, wherein said polypyranose is hydroxyethyl starch.

10. The lens of claim 6, wherein said polypyranose is a glycosaminoglycan.

11. The lens of claim 10, wherein said glycosaminoglycan is hyaluronic acid.

12. The lens of claim 10, wherein said glycosaminoglycan is heparin.

13. The lens of claim 10, wherein said glycosaminoglycan is chondroitin sulfate.

14. The lens of claim 1, wherein said lipid or phospholipid moiety bound to a physiologically acceptable monomer, dimer, oligomer, or polymer is coated on at least a portion of a surface of said contact lens.

15. The lens of claim 1, wherein said lipid or phospholipid moiety bound to a physiologically acceptable monomer, dimer, oligomer, or polymer is embedded in at least a portion of said contact lens.

16. The lens of claim 1, wherein the lipid or phospholipid moiety bound optionally via a spacer to a physiologically acceptable monomer, dimer, oligomer, or polymer via an ester or amide bond, and/or a pharmaceutically acceptable salt or a pharmaceutical product thereof is represented by the structure of the general formula (A): 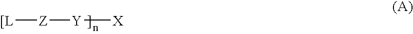
wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a physiologically acceptable monomer, dimer, oligomer, or polymer; and
n is a number from 2 to 1000;
wherein any bond between L, Z, Y and X is either an amide or an esteric bond.

17. The lens of claim 16, L is phosphatidyl, Z is ethanolamine, Y is nothing, and X is carboxymethylcellulose or a glycosaminoglycan.

18. The lens of claim 16, wherein the phosphatidylethanolamine moiety is dipalmitoyl or dimyristoyl phosphatidylethanolamine.

19. The lens of claim 16, wherein the lipid or phospholipid moiety bound optionally via a spacer to a physiologically acceptable monomer, dimer, oligomer, or polymer via an ester or amide bond, and/or a pharmaceutically acceptable salt or a pharmaceutical product thereof is represented by the structure of the general formula (I): 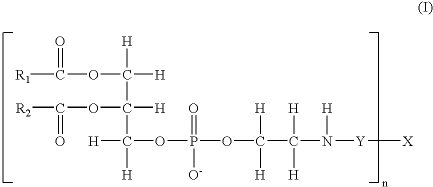
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms; and
Y is either nothing or a spacer group ranging in length from 2 to 30 atoms;
X is a physiologically acceptable monomer, dimer, oligomer, or polymer, and wherein X is a glycosaminoglycan; and
n is a number from 1 to 1000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

20. The lens of claim 16, n is a number from 2 to 100.

21. The lens of claim 16, wherein X is hyaluronic acid, heparin or chondroitin sulfate.

22. A method of treating an ocular surface disease in a subject, the method comprising applying a lens of claim 1 to the eye of a subject.

23. The method of claim 22, wherein said disease is dry eye or Keratoconus.
